(19) United States
(12) Patent Application Publication
(10) Pub. No.: US 2005/0143641 A1
(43) Pub. Date: Jun. 30, 2005

(54) Medical information processing system

(75) Inventor: Koichi Tashiro, Sagamihara-shi (JP)

Correspondence Address: Thomas Spinelli;Scully, Scott, Murphy & Presser, Suite 300, 400 Garden City, Garden City, NY, 11530, US

(73) Assignee: Olympus Corporation, Tokyo (JP)

(21) Appl. No.: 11/018,599

(22) Filed: Dec. 21, 2004

(30) Foreign Application Priority Data
Dec. 25, 2003 (JP) 2003-431381

(51) Int. Cl. A61B 5/05

(52) U.S. Cl. 600/407; 600/410

(57) ABSTRACT

Additional information (for example, inclination of gantry/detector of image formation apparatus, and slice positions of tomographic images) is added to medical images, and the medical images are stored in an image storage unit. Based upon the additional information, a sequence for displaying the medical images on a viewer is determined. The medical images are displayed in accordance with this determined sequence. As a result, there are larger possibilities that resembling medical images are displayed and positioned close to each other on the viewer and a diagnosing operation by a doctor can be easily carried out.

### CROSS-REFERENCE TO RELATED APPLICATION

This application is based upon and claims the benefit of priority from the prior Japanese Patent Application No. 2003-431381, filed Dec. 25, 2004, the entire contents of which are incorporated herein by reference.

### BACKGROUND OF THE INVENTION

1. Field of the Invention

The present invention is related to a medical information processing system for displaying a plurality of medical images acquired by imaging the same object.

2. Description of the Related Art

In imaging apparatus known such as CT (computerized tomographic) and MRI (magnetic resonance imaging), file names and date of formation information of their medical images have been applied independently. Viewers such as a DICOM viewer can display these medical images, and can rearrange the display sequence of these medical images in ascending or descending order based upon the file names and the image date of formation information.

Since doctors try to find slight differences of these medical images such as tomographic images and identify diseased portions by visually comparing them, the displaying sequence of these medical images is important.

However, even if medical images taken by imaging apparatus, such as CT or MRI, are arranged based on the order of file names and/or date of formation information of image files, these medical images are not always displayed in such a sequence that adjacent images resemble each other.

For example, in the field of brain surgeries, a total number of tomographic images may exceed 200 in one case. Therefore, if medical images are arranged in a disorderly fashion irrespective of resemblance, a long time is required to find and retrieve necessary medical images.

### BRIEF SUMMARY OF THE INVENTION

A medical information processing system of the present invention, belonging to such a system for displaying a plurality of medical images taken from the same object, identifies features of the each medical image based on additional information added to each medical image and determines the sequence of displaying these images based on the result of the identification.

Preferably, the additional information contains at least one of patient relative information and information indicative of an image formation condition. If the display sequence of the medical images is determined by employing the patient relative information and the image formation condition, then there are larger possibilities that medical images having high resemblance are displayed and positioned close to each other. Examples of the patient relative information and the image formation condition includes, but is not limited to, an inclination of either a gantry or a detector of an image formation apparatus, and a slice position in a case that a medical image corresponds to a tomographic image.

When such medical images that inclinations of either the gantry or the detectors of the image formation apparatus are equal or similar to each other are arrayed, since imaging angles of these arrayed medical images are resembled to each other, there are larger possibilities that the resembling medical images are displayed and positioned close to each other. In the same meaning, when such medical images that slice positions thereof are equal or similar to each other are arrayed, since medical images of the same or similar portions are arrayed, there are larger possibilities that the resembling medical images are displayed and positioned close to each other.

The present invention is effective in such a case that medical images acquired by plural sorts of medical image formation apparatus are displayed by employing one viewer.

There are many opportunities that titles of medical images are named in relation to names of patients and IDs of patients, or numerically named by using sequence numbers. Therefore, there are many cases that even modalities are different from each other, titles of medical images are resembled to each other. As a consequence, in particular, the present invention is useful in the case that a plural of medical image formation apparatus give similar names to images they take and discrimination of these images is difficult based on their names. However, this invention does not always require the existence of the plural sorts of medical image formation apparatus, but the present invention may be alternatively applied to such a case that a plurality of medical images formed by one or one sort of medical image formation apparatus are displayed.

As previously explained, when the arranging sequence of the medical images is determined on the viewer side based upon the additional information, a degree of freedom and independence for applying file names is increased.

Preferably, additional information is positioned in front of a medical image in a medical image data file. When such a file configuration is employed, only the additional information may be read without reading a medical image having a large amount of data. As a result, an access speed for determining a display sequence may be increased.

In the same logic, it is also possible to store the additional information and the main body of the medical image data separately from each other to increase the access speed.

Preferably, a portion for storing the additional information is divided into a plurality of data items. In this data structure, plural sorts of additional information can be stored, and thus a variation for determining display sequence of the medical images is increased.

In the present invention, the display sequence of medical images is determined based upon the patient relative information and/or the image formation condition of the object under examination thereof, and the medical images are displayed in accordance with the determined display sequence. As a consequence, the medical images that are resembled to each other may be readily displayed and positioned close to each other.

The present invention is useful for a diagnostic operation, for instance, a doctor visually compares slight differences between respective medical images with each other so as to specify a shape of a diseased portion.

### BRIEF DESCRIPTION OF THE SEVERAL VIEWS OF THE DRAWINGS

These and other features, aspects, and advantages of the apparatus and methods of the present invention will become better understood with regard to the following description, appended claims, and accompanying drawings where:

FIG. 1 is a structural diagram for representing an arrangement of a medical information processing system according to an embodiment of the present invention;

FIG. 2 is a block diagram for schematically showing an arrangement of a controller of FIG. 1;

FIG. 3 is a first diagram for explaining display sequence of medical reference images which are displayed in a reference image display area on a display screen of a display apparatus of FIG. 1;

FIG. 4 is a second diagram for explaining display sequence of medical reference images which are displayed in the reference image display area on the display screen of the display apparatus of FIG. 1;

FIG. 5 is a first diagram for explaining a relationship in the sequence between patient relative information contained in the medical reference image of FIG. 3 and/or image formation conditions, and the medical reference images;

FIG. 6 is a second diagram for explaining a relationship in the sequence between patient relative information contained in the medical reference image of FIG. 3 and/or image formation conditions, and the medical reference images;

FIG. 7 is a diagram for explaining a structure of a data file which contains the patient relative information and/or the information indicative of the image formation conditions of FIG. 3;

FIG. 8 is a flow chart for describing a flow of a display sequence determining process of the medical reference images executed by a CPU of FIG. 2;

FIG. 9 is a diagram for explaining a structure of a modification of the data file which contains the patient relative information and/or the information indicative of the image formation conditions of FIG. 7; and

FIG. 10 is a block diagram for schematically indicating an arrangement of a modification of the controller of FIG. 1.

### DETAILED DESCRIPTION OF THE INVENTION

Preferred embodiments of the invention will be described below with reference to the accompanying drawings.

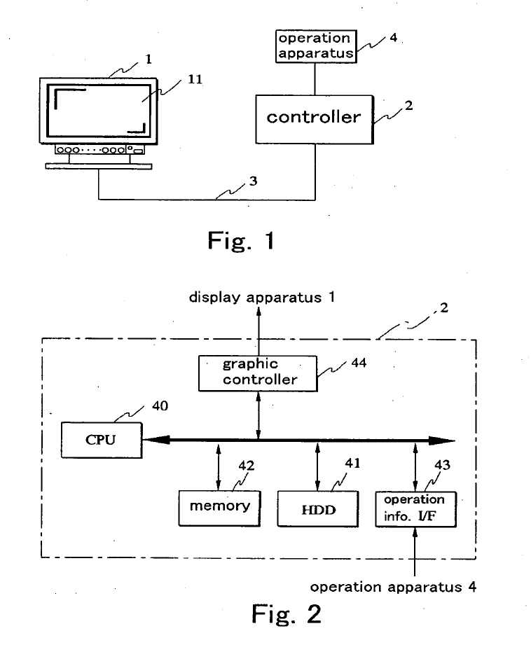

FIGS. 1-10 are related to an embodiment of the present invention.

As represented in FIG. 1, in the medical information processing system of this embodiment, a display apparatus 1 is connected to a controller 2 with a cable 3. The controller 2 contains an operation apparatus 4 such as a keyboard, or a mouse, which controls displaying function. The cable 3 such as a VGA cable conveys image signals.

FIG. 2 indicates an internal arrangement of the above-described controller 2. The controller 2 is equipped with an operation information I/F (interface) 43, a HDD (hard disk drive) 41 and/or a memory 42, and a CPU (central processing unit) 40. The operation information I/F 43 acquires operation information from the operation apparatus 4. The HDD 41 and/or the memory 42 correspond to an image storage unit for storing image data. The CPU 40 corresponds to an operating device functioning as an image characteristic identifying unit and a display control unit. The image characteristic identifying unit identifies characteristics of medical images based on such information as patient relative information and/or image formation conditions of an object under examination, which corresponds to additional information included in or otherwise associated with the images. The CPU 40 determines sequence of display based upon the additional information. The controller 2 further contains a graphic controller 44, while the graphic controller 44 reads out an image stored in the HDD 41 and/or the memory 42, and then, displays the read image on the display apparatus 1.

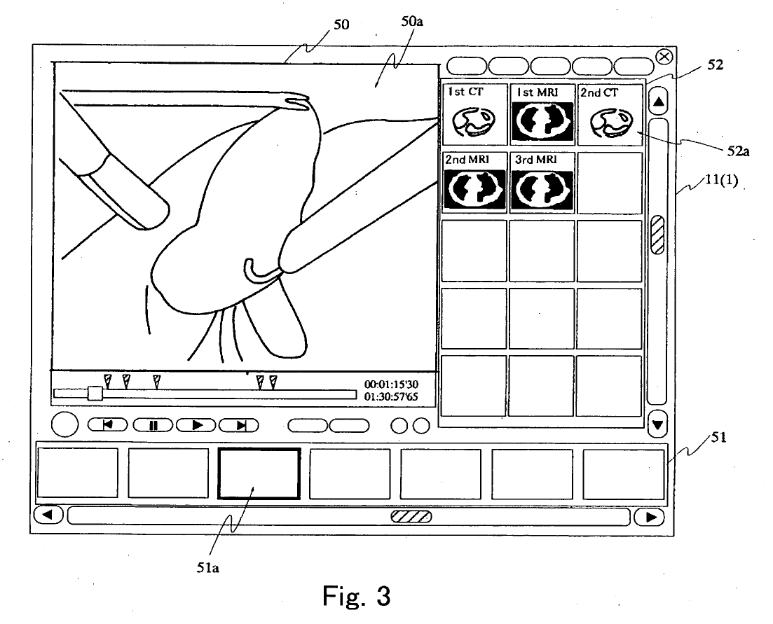

As indicated in FIG. 3, a display screen 11 of the display apparatus 1 has a medical image display area 50, a thumbnail display area 51, and a reference image display area 52.

On the display screen 11 of the display apparatus 1, for instance, a medical moving image 50a acquired at a medical examination with an endoscope is displayed in the medical image display area 50. The data of the medical moving image 50a has been stored in the HDD 41 and/or the memory 42. Thumbnail images 51a are displayed in the thumbnail display area 51. The thumbnail images 51a are constituted from a plurality of still images such as frame of interest in the medical moving image 50a.

Also, at least one medical reference image (X-ray image, ultrasonic image, CT image, MRI image etc.) 52a of a patient corresponding to an examination subject is displayed in the reference image display area 52, while the data of the medical reference image 52a has been stored in the HDD 41 and/or the memory 42.

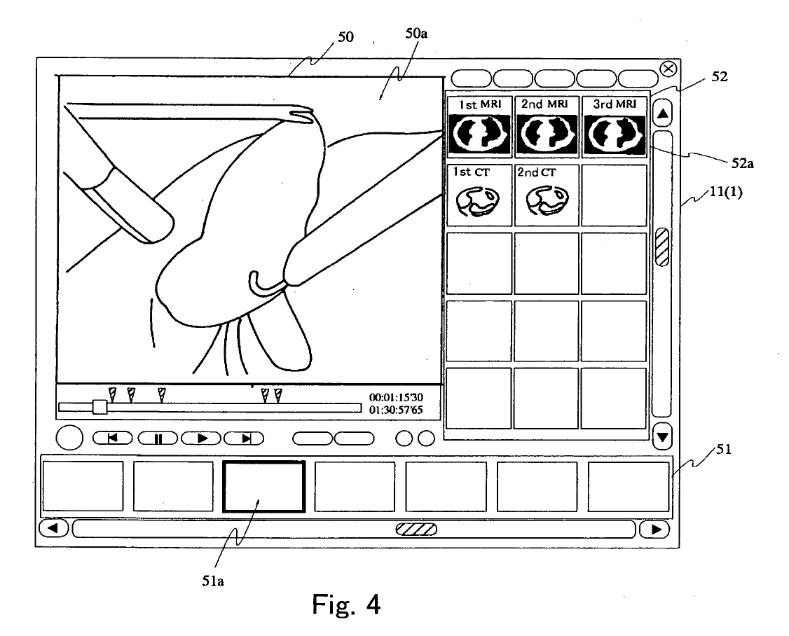

In a display control for the medical reference image 52a displayed in the reference image display area 52, the patient relative information and/or the image formation conditions of the patient corresponding to the examination subject are evaluated by the CPU 40 so as to determine sequence of displays, and thus, the medical reference images 52a are displayed in such a sequence different from that of FIG. 3. FIG. 4 shows an example in which the medical reference image 52a is displayed in sequence different from that of FIG. 3.

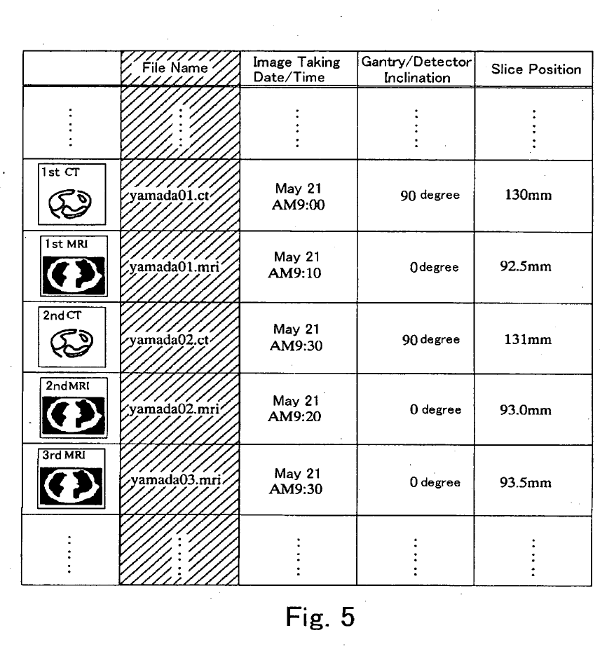

FIG. 5 shows a portion of information items that are added to the medical reference image 52a displayed in the reference image display area 52. The information items contain a file name, image formation date/time, an inclination of a gantry/detector, and a slice position. FIG. 5 shows an example that the medical reference images 52a are arranged according to the order of the file names. This image arrangement corresponds to the display manner of FIG. 3.

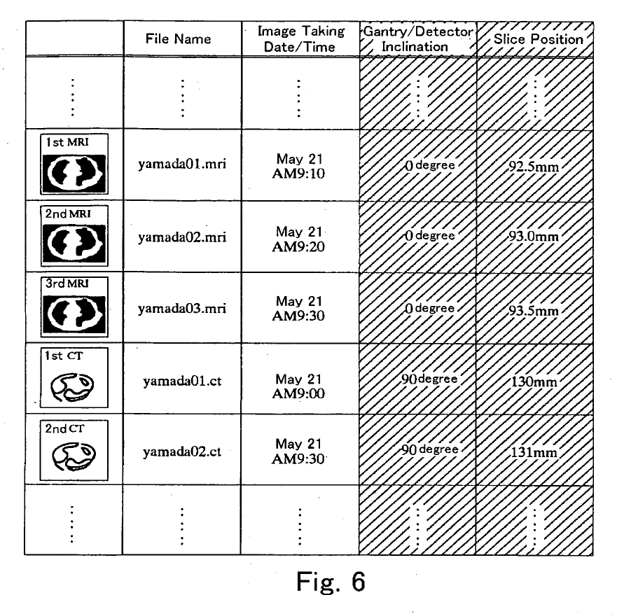

Similarly, FIG. 6 shows a display sequence of the medical reference images 52a in such a case that the gantry/detector inclination and the slice position are set as references and the medical reference images 52a are rearranged in an ascent order thereof. This image arrangement manner corresponds to the display manner of FIG. 4.

In the embodiment, a term of “gantry/detector inclination” implies detection data of a detector which detects an inclination of a gantry on which a patient is laid when an imaging operation is carried out for taking an X-ray image, an ultrasonic image, a CT image, an MRI image, and the like. Also, a term of “slice position” corresponds to section slice positional information in a CT image and MRI image.

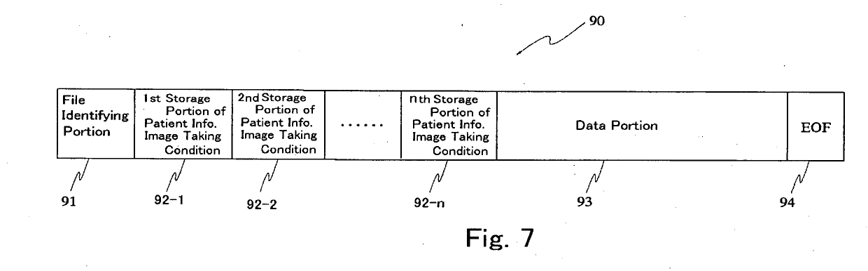

FIG. 7 indicates a file structure of a data file 90 of the medical image. At least, one piece of the information such as image formation date/time, and gantry/detector inclinations has been stored in “i”-th storage portions 91-i of patient information/image formation conditions of the data file 90. It should be noted that symbol “i” can take one value between 1 to “n.”

In this data file 90, a file-identifying portion 91 indicative of a head of the file is present. This data file 90 represents that the file identifying portion 91 is positioned at the head portion of this data file 90; the “i”-th storage portions 92-i for patient information and/or image formation conditions succeed the file identifying portion 91; a data portion 93 into which image information such as an organ, CT, and MRI is stored, succeeds the “i”-th storage portions 92-i; and an EOF (End of File) 94 indicates an end of the data file 90.

Next, operations of the medical information processing system according to this embodiment will be described. The HDD 41 and/or the memory 42 contained inside the controller 2 has stored therein the image data such as the medical moving image 50a and the medical reference images 52a, while this controller 2 is connected to the display apparatus 1 with the cable 3 such as the VGA cable for carrying the picture signals. The CPU 40 identifies one or more characteristics of the images based the patient relative information and/or the image formation condition, which are the additional information added to the medical reference image 52a.

As indicated in FIG. 3, the display screen 11 displays thereon the images which are constituted by the medical image display area 50, the thumbnail display area 51, and the reference image display area 52. More specifically, at least one of the medical reference image 52a which have been stored in the HDD 41 and/or the memory 42 is arranged to be displayed in the reference image display area 52.

In the case that the medical reference images 52a are displayed randomly in the reference image display area 52, as shown in FIG. 3, the medical reference images 52a are displayed irrespective of resemblance characteristics thereof and relative relationships therebetween. On the other hand, as shown in FIG. 4, the CPU 40 determines sequence of displays based upon at least one of the patient relative information and/or the image formation conditions of the object attached to the images, and the graphic controller 44 reads out the images which have been stored in the HDD 41 and/or the memory 42, and then, the medical reference images 52a are displayed in the reference image display area 52 in accordance with the sequence determined by the CPU 40.

In another way, for instance, as shown in FIG. 5, in the case that the medical reference images 52a are arranged in accordance with their file names, different kind of medical reference images 52a may be mixed with each other. On the other hand, as represented in FIG. 6, the CPU 40 rearranges these medical reference images 52a by using the gantry/detector inclination and the slice position as the reference. Therefore a doctor can readily and visually compare the medical reference images 52a with each other, and the doctor can notice a slight difference between resembling images so as to specify a pathological change.

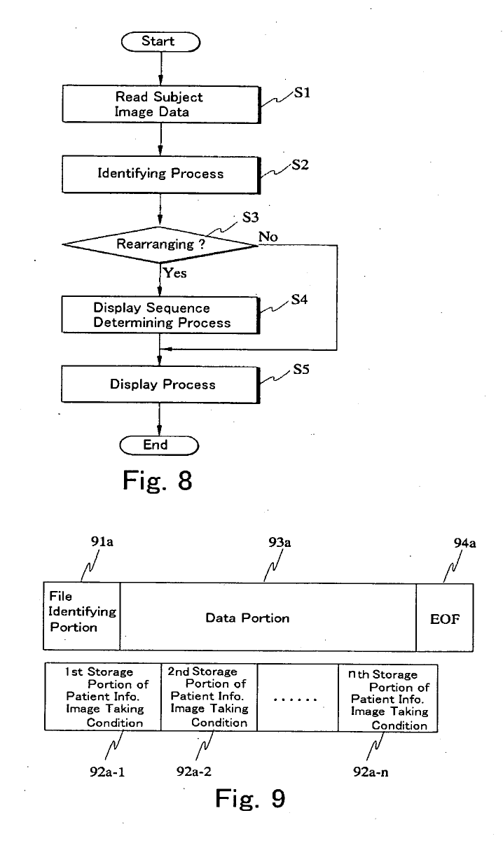

A rearranging process for the medical reference images 52a is executed as indicated in the flow chart of FIG. 8. That is, in a step S1 of the flow chart, the CPU 40 reads subject image data. In a step S2, which is an identifying process of an image characteristic, the CPU 40 identifies the patient information and/or image formation conditions of the object under examination, which are added to the medical reference image 52a.

At step S3, the CPU 40 determines whether or not the rearranging process is carried out based upon the identified information. In the case that the rearranging process is not carried out (S3-No), the CPU 40 displays the medical reference images 52a in the original display sequence in a display process of step S5. In the case that the rearranging process is carried out (S3-Yes), the CPU 40 determines display sequence in a display sequence determining process of the step S4. Thereafter, in the display process of the step S5, the CPU 40 displays the medical reference images 52a in accordance with the display sequence determined in the display sequence determining process of the step S4.

As indicated in FIG. 7, the data file 90 contains at least one of the patient relative information and image formation condition storage portions 92-i. The rearranging process is carried out by the CPU 40 based upon the patient relative information and/or the image formation condition of the object under examination, which have been identified.

As an alternative data configuration, shown in FIG. 9, the “i”-th storage portions 92a-i (“i” corresponds one of 1 to n) for patient relative information and/or image formation conditions may be separated from a file identifying portion 91a, a data portion 93a, and an EOF 94a. In such a case, the “i”-th storage portions 92a-i for patient relative information and/or image formation condition may be related to the other part of the data structure in the controller 2, and such relations may be retained in the controller 2. In FIG. 9, the “i”-th storage portions 92a-i for patient information and/or image formation conditions are managed on the side of the operating system as such the “i”-th storage portions 92a-i have been stored with relations to such data which is commenced by the file identifying portion 91a.

Generally speaking, the data portion 93a of the data file 90 has a large data capacity. More specifically, in the case that there are large numbers of images stored in the data portion 93a of the data file 90, a heavy workload is given in order to process these images by the CPU 40. Therefore, in the example shown in FIG. 9, by separately storing the “i”-th storage portions 92-i for patient information/image formation conditions, the CPU 40 can quickly determine display sequence without reading the whole part of data but just reading the separated portion of it.

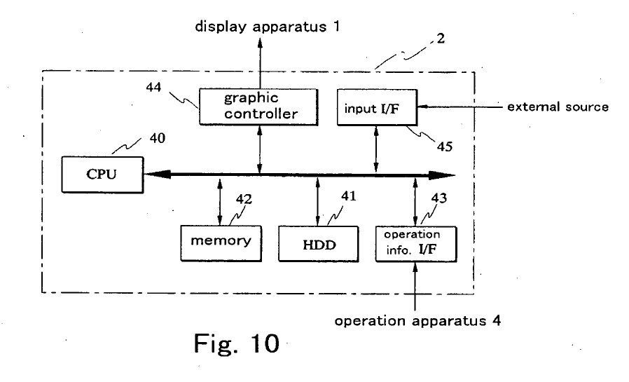

It should also be noted that as shown in FIG. 10, an input I/F 45 for inputting the medical reference images 52a may be alternatively provided within the controller 2. In this alternative case, in the controller 2, an image input signal which is entered via the input I/F 45 from an external source is processed by the graphic controller 44 and displayed on the display apparatus 1. Further, the medical reference images 52a derived from the external source are stored in the HDD 41 and/or the memory 42. Therefore, the medical reference images 52a that have been entered from the external source may be alternatively stored and displayed.

In the embodiment, the medical information processing system analyzes the patient relative information and/or the image formation conditions of the object under examination, and identifies the characteristics of the corresponding images based thereon, and determines the sequence of the displays based upon the identification result, and then, displays the medical reference images in accordance with the determined display sequence. As a consequence, even in such a case that when the display sequence is determined by identifying the image names and the image forming dates/times and such of the display sequence gives poor resemblance between neighboring images, this system of the embodiment can rearrange display sequence with more resemblance. When the medical images are arranged by identifying, for instance, the inclinations of either the gantries or the detectors of the image formation apparatus that are identical or similar to each other, then there are larger possibilities that the resembling medical images are to be displayed and positioned close to each other. As a result, while the doctor visually compares these medical images with each other, the doctor can readily diagnose these medical images, for instance, can easily specify shapes of diseased portions. Also, the doctor can retrieve the necessary medical reference images in a higher efficiency.

Also, by arranging medical images where the inclinations of either the gantry or the detector of the image formation apparatus are equal or resembled to each other without depending upon the formation day/times, medical practitioners can enjoy easy grasping of the pathological changing portion of the same patient in the lapse of time after the medical operation.

Furthermore, when data of elapsed time after the medical operation is added to in the image data, by rearranging the pictures in ascending or descending order from the operation time, the medical practitioners can readily realize the recovering processes.

When medical history information such as the treatment times and the dosage times is contained as the additional information, the medical images can be rearranged in either ascending order or descending order based on such information. As a result, the recovery effect achieved by the dosage/treatment can be grasped, at the same time, some side effects or complications incurred on another organ by the treatment or dosage can be detected.

In curing of adiposis, the U.S.A has a large number of patients thereof, therefore, patient weight-related information such as weight, Body Mass Index, body fat amount can be used as the additional information. In such a case, the medical images can be arranged in either ascending order or descending order based upon the patient weight-related information. Such a display sequence may be useful to grasp increases/decreases in a particular amount and an organ fat amount, and to diagnose the risk of diabetes, hypertension, hyperlipemia, and the like.

When age information is contained as the additional information, the medical images can be arranged in a time sequential manner even in such a case that a modality's machine time is erroneously set and the medical images taken by the modality have incorrect time information.

In addition, the above-explained rearranging methods based upon the additional information may be effectively applied not only to the image comparing/investigating operation for the same patient, but also to the image comparing/investigating operations with another patient.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

1. A medical information processing system for displaying a plurality of images acquired by imaging an object, the system comprising:
an image storage unit for storing images of the object to which additional information has been added, the additional information including at least one of patient relative information and information indicative of an image formation condition;
an image characteristic identifying unit for identifying a characteristic of the images based upon the additional information; and
a display control unit for determining a display sequence of the images based upon an identification result by the image characteristic identifying unit.

2. A medical information processing system as claimed in claim 1, further comprising:
an image input unit for inputting the images from an external source.

3. A medical information processing system as claimed in claim 1 wherein:
the images contain at least one of a CT image and an MRI image of the object; and
the patient relative information and/or the image formation condition contain slice section positional information of the object of the CT image and/or the MRI image.

4. A medical information processing system as claimed in claim 1 wherein:
the additional information includes an inclination of at least one of a gantry and a detector of an image formation apparatus.

5. A medical information processing system as claimed in claim 1 wherein:
the additional information includes time elapsed after a medical operation.

6. A medical information processing system as claimed in claim 1 wherein:
the additional information corresponds to information containing a patient treatment history of at least one of curing times and dosage times.

7. A medical information processing system as claimed in claim 1 wherein:
the additional information includes patient weight-related information containing at least one of weight, Body Mass Index, and body fat amount.

8. A medical information processing system as claimed in claim 1 wherein:
the additional information includes age of a patient.

9. A medical information processing system as claimed in claim 1 wherein:
the image storage unit is capable of storing a plurality of patients' image information with the additional information;
the image characteristic identifying unit is capable of identifying the additional information for each of the plurality of patients; and
the display control unit is capable of determining the display sequence of the images for each of the patients based upon the identification result by the image characteristic identifying unit.

10. A medical information processing system as claimed in claim 1, further comprising means for varying the identification of the characteristic.

11. A medical information processing system comprising:
an image storage unit for storing a plurality of medical images to which additional information has been added;
a calculation device for performing a calculation based upon at least some of the additional information so as to determine a display sequence of the plurality of medical images; and
a display for displaying at least some of the plurality of the medical images in accordance with the display sequence determined by the calculation device.

12. A medical information processing system as claimed in claim 11 wherein:
the image storage unit has a data structure of at least some of the plurality of medical images in which the additional information is positioned in front of the medical image data.

13. A medical information processing system as claimed in claim 11 wherein:
the additional information comprises a plurality of data items.

14. A medical information processing system as claimed in claim 11 wherein:
the additional information is stored separately from a corresponding one of the plurality of medical images.

15. A medical information processing system as claimed in claim 11 wherein:
the additional information contains an inclination of at least one of a gantry and detector of an image formation apparatus.

16. A medical information processing system as claimed in claim 11 wherein:
the medical images includes a tomographic image; and the additional information contains a slice position of the tomographic image.

17. A medical information processing system as claimed in claim 11 wherein:
the additional information includes time elapsed after a medical operation.

18. A medical information processing system as claimed in claim 11 wherein:
the additional information includes a treatment history of a patient comprising at least one of a number of times for medical treatments and dosages.

19. A medical information processing system as claimed in claim 11 wherein:
the additional information includes patient weight-related information including at least one of weight, Body Mass Index, and body fat amount.

20. A medical information processing system as claimed in claim 11 wherein:
the additional information includes age of a patient.

21. A medical information processing system as claimed in claim 11 wherein:
the image storage unit is capable of storing a plurality of patients' image information with the additional information;
the calculation device is capable of identifying the additional information for each of the plurality of patients; and
the display is capable of determining the display sequence of the plurality of images based upon the identification result made by the calculation device for each of the plurality of patients.

22. A medical information processing method wherein:
reading a plurality of medical images from an image storing unit for storing the plurality of medical images, at least some of the plurality of medical images having additional information;
determining a sequence of display for the at least some of the plurality of medical images based on the additional information; and
displaying the plurality of medical images according to the determining step.
